(19) United States
(12) Patent Application Publication
(10) Pub. No.: US 2026/0327307 A1
(43) Pub. Date: Oct. 22, 2026

(54) PRO-ANTIBODY THAT REDUCES OFF-TARGET TOXICITY

(71) Applicant: IMMUNELOGIC THERAPEUTICS, INC., Beijing (CN)

(72) Inventor: Kuo-Fu Tseng, Dallas, TX (US)

(73) Assignee: IMMUNELOGIC THERAPEUTICS, INC., Beijing (CN)

(21) Appl. No.: 19/694,170

(22) Filed: Jun. 1, 2026

Related U.S. Application Data
(62) Division of application No. 17/150,626, filed on Jan. 15, 2021, now Pat. No. 12,662,535.
(60) Provisional application No. 62/962,555, filed on Jan. 17, 2020.

(51) Int. Cl. C07K 16/28; A61K 35/17; A61K 45/06

(52) U.S. Cl. CPC C07K 16/2809; A61K 35/17; A61K 45/06; C07K 16/2878; C07K 2317/56; C07K 2319/03; C07K 2319/30; C07K 2319/50

(57) ABSTRACT

The present invention includes proteins, nucleic acids and methods of making and using an activatable antibody (aAb) comprising, in order, the following structure: a first light chain comprising: a first variable light region; a cleavable linker; a first heavy chain comprising: a first variable heavy region; wherein the cleavable linker prevents or reduces the first light chain and the first heavy chain from forming a first antigen binding site against a first antigen; and wherein cleavage of the cleavable linker releases the first heavy chain to allow formation of the first antigen binding site to bind a first antigen.

### CROSS-REFERENCE TO RELATED APPLICATIONS

This application is a divisional of U.S. application Ser. No. 17/150,626, filed Jan. 15, 2021, which claims priority to U.S. Provisional Application Ser. No. 62/962,555, filed Jan. 17, 2020, the entire contents of which are incorporated herein by reference.

### TECHNICAL FIELD OF THE INVENTION

The present invention relates in general to the field of pro-antibodies (probodies) that reduce normal tissue targeting and that enhance tumor targeting.

### STATEMENT OF FEDERALLY FUNDED RESEARCH

Not applicable.

### REFERENCE TO A SEQUENCE LISTING

The present application includes a Sequence Listing. The contents of the electronic sequence listing (AttachE_Sequence_Listing-0198.xml; Size: 6,865 bytes; and Date of Creation: May 29, 2026) is herein incorporated by reference in its entirety.

### BACKGROUND OF THE INVENTION

Without limiting the scope of the invention, its background is described in connection with probodies, or activatable antibodies.

One such probody is taught in U.S. Pat. No. 10,059,762, issued to Stagliano, et al., entitled, “Anti-EGFR activatable antibodies”. These inventors teach modified antibodies that contain an antibody or antibody fragment (AB) modified with a masking moiety (MM), which can be further coupled to a cleavable moiety (CM), resulting in activatable antibodies (AAs). In the AA, the CM is capable of being cleaved, reduced, photolyzed, or otherwise modified. AAs can exhibit an activatable conformation such that the AB is more accessible to a target after, for example, removal of the MM by cleavage, reduction, or photolysis of the CM in the presence of an agent capable of cleaving, reducing, or photolyzing the CM. However, a significant limitation of this art is the competition between the MM and the target for binding to the target binding moiety (TBM). Further, cleavage of the MM leads to significant off-target effects.

Another such probody is taught in U.S. Pat. No. 10,233,244, issued to Sagert, et al., entitled, “Anti-ITGA3 antibodies, activatable anti-ITGA3 antibodies, and methods of use thereof”. The invention is said to relate generally to antibodies that bind ITGa3, activatable antibodies that specifically bind to ITGa3 and methods of making and using these anti-ITGa3 antibodies and anti-ITGa3 activatable antibodies in a variety of therapeutic, diagnostic and prophylactic indications.

Another such probody is taught in U.S. Pat. No. 8,541,203, issued to Daugherty, et al., entitled, “Activatable binding polypeptides and methods of identification and use thereof”. These inventors teach activatable binding polypeptides (ABPs), which contain a target binding moiety (TBM), a masking moiety (MM), and a cleavable moiety (CM). The masking moiety covers the cognate binding site of the TBM, and the TBM is exposed upon cleavage of the CM. Certain activatable antibody compositions are said to include a TBM containing an antigen binding domain (ABD), a MM and a CM. The ABPs are said to include an “activatable” conformation such that at least one of the TBMs is less accessible to target when uncleaved than after cleavage of the CM in the presence of a cleaving agent capable of cleaving the CM. The application is said to teach libraries of candidate ABPs, methods of screening to identify such ABPs, and methods of use. ABPs specific for VEGF, CTLA-4, or VCAM are taught with ABPs having a first TBM that binds VEGF and a second TBM that binds FGF, as well as compositions and methods of use, at taught. Again, a significant limitation of this art is the competition between the MM and the target for binding to the TBM. Further, cleavage of the MM leads to significant off-target effects.

Another such probody is taught in U.S. Patent Publication No. 20190359714, filed by Tipton, et al., “Activatable Anti-CTLA-4 Antibodies and Uses Thereof”. These applicants are said to teach activatable anti-human CTLA-4 antibodies comprising a heavy chain comprising a VH domain and a light chain comprising a masking moiety (MM), a cleavable moiety (CM), and a VL domain. Such activatable anti-human CTLA-4 antibodies have CTLA-4 binding activity in the tumor microenvironment, where the masking moiety is removed by proteolytic cleavage of the cleavable moiety by tumor-specific proteases, but exhibit greatly reduced binding to CTLA-4 outside the tumor.

Another such probody is taught in U.S. Patent Publication No. 20180271997, filed by Wang, entitled, “Methods and Reagents to Treat Tumor and Cancer”. This applicant is said to teach reagents to treat tumor and cancer, and methods of using the same for treating tumor and cancer using a pro-antibody, which is antibody that can be activated in tumor. Another type of the reagent is said to be a conjugate of sialidase with affinity ligand that can bind to an immune cell surface or a conjugate of sialidase with affinity ligand that can bind to another antibody, therefore provide a sialidase based cancer immunotherapy.

Three approaches for activatable antibodies have been used to date. In the first, the probody includes a masking peptide linked via a proteolytic linker to block the antigen-antibody interaction. The development of each probody requires a screening process using phage display for a masking peptide. After cutting of linker, the masking peptide is expected to go away and release the antigen binding site of antibody. The problems with this approach include: (1) the heterogeneity of masking peptide itself induces immune responses; (2) the failure to release the masking peptide reduces efficacy; (3) some antigen-antibody interactions are too strong to be masked by a short (e.g., 10 amino acid) peptide; and (4) the peptides can be degraded before reaching to tumors and expose toxicity of antibodies to normal tissues.

In the second approach, Dual Variable Domain Immunoglobulins (DVD-Ig) are said to reduce the toxicity of anti-CTLA4 antibody. The VL and VH of an anti-TTA (Tumor Targeting Antigen) antibody are linked to the anti-CTLA-4 antibody via proteolytic linkers to cover the CTLA4 binding site. Unfortunately, this approach requires the pairing of the tumor associated antigen (TAA) and CTLA4, it requires adding an exact set of VL and VH, thus making the molecule bigger, and the exact VL and VH can always interact with off-target antigen and induce immune responses.

The third approach physically blocks the binding of an anti-CD3 variable light (VL) chain and a variable heavy (VH) by linking a “pseudo VL” and “pseudo VH” pair to an active and functional VL and VH. When pseudo VL interacts with VH, and when the pseudo VH interacts with VL, it does not have activity. Protease linkers are used to link together VL, VH, pseudo-VL and pseudo-VH, creating a very large molecule.

What is needed are novel fusion proteins that overcome the problems in the prior art, by eliminating targeting of normal tissues and enhancing the activity at the tumor target site.

### SUMMARY OF THE INVENTION

In one embodiment, the present invention includes an activatable antibody (aAb) comprising, in order, the following structure: a first light chain comprising: a first variable light region; a cleavable linker; a first heavy chain comprising: a first variable heavy region; wherein the cleavable linker prevents or reduces the first light chain and the first heavy chain from forming a first antigen binding site against a first antigen; and wherein cleavage of the cleavable linker releases the first heavy chain to allow formation of the first antigen binding site to bind a first antigen. In one aspect, the aAb further comprises a second antibody binding site formed by a second variable light chain and a second constant light chain connected to the first heavy chain that binds a second antigen, and optionally a flexible non-cleavable linker between the second variable light chain and the second constant light chain. In another aspect, the aAb further comprises at least one of a first constant heavy region, a first constant heavy region, or both. In another aspect, the first light chain region, the first heavy chain region, or both, further comprise an Fc region, a wild-type Fc region, a mutated Fc region, a monomeric wild type Fc region, a monomeric mutant Fc region, a dimeric wild type Fc region, a dimeric mutant Fc region, a second variable heavy region and a second Fc region, or a second variable heavy region and a second Fc region and an uncleavable flexible linker and a second variable light region and a second heavy variable region, or a second Fc region and an uncleavable flexible linker and a cytokine. In another aspect, the first and second antigen are at least one of: the same antigen; the first and second antigen are different; or the first and second antigen is the same antigen but the first antigen binding site and the second antigen binding site bind different epitopes of the same antigen. In another aspect, the first antigen binding site or the second antigen binding site binds a tumor target. In another aspect, the first antigen is a tissue specific surface antigen selected from ICAM1; VCAM1; EpCAM; extra domain B of fibronectin; melanoma-associated chondroitin sulfate proteoglycan (MCSP); melanoma-associated proteoglycan (MAPG); high molecular weight melanoma associated antigen (HMV-MAA); prostate specific membrane antigen (PSMA); epidermal growth factor receptor (EGFR); hepatocyte growth factor receptor (HGFR); fibroblast activation protein (FAP); carcinoembryonic antigen (CEA); cell-adhesion molecule (CAM); human B-cell maturation target (BCMA); placental growth factor (PLGF); folate receptor, insulin-like growth factor receptor (ILGFR); CD133; CD40; CD37; CD33; CD30; CD28; CD24; CD23; CD22; CD21; CD20; CD19; CD13; CD10; HER3; HER2; nonmuscle myosin heavy chain type A (nmMHCA); transferrin; epithelial cell adhesion molecule (EpCAM); annexin A 1; nucleotin, tenascin, vascular endothelial growth factor receptor 1 (VEGFR1), vascular endothelial growth factor receptor 2; (VEGFR-2); aminopeptidase N, tie-1, tie-2, or c-Met. In another aspect, the first antigen is selected from a protein, a portion of a protein, or a peptides encoded by at least one gene selected from: ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1; B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-la); CCL4 (MIP-lb); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR 13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wapl/Cipl); CDKNIB (p27Kipl); CDKNIC; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CXCLIO(IP-IO); CXCL11 (I-TAC/IP-9); CXCL12 (SDF1); CXCL13; CXCL14; CXCL16; CXCL2 (GR02); CXCL3 (GR03); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; CGRP; Clq; Clr; CI; C4a; C4b; C2a; C2b; C3a; C3b; DAB2IP; DES; DKFZp451J0118; DNCL1; DPP4; E-selectin; E2F1; ECGF1; EDG1; EFNA1; EFNA3; EFNB2; EGF; EGFR; ELAC2; ENG; ENOl; EN02; EN03; EPHB4; EPO; ERBB2 (Her-2); EREG; ERK8; ESR1; ESR2; F3 (TF); Factor VII; Factor IX; Factor V; Factor Vila; Factor Factor X; Factor XII; Factor XIII; FADD; FasL; FASN; FCER1A; FCER2; Fc gamma receptor; FCGR3A; FGF; FGFl (aFGF); FGF10; FGFll; FGF12; FGF12B; FGFl 3; FGFl 4; FGF16; FGFl 7; FGFl 8; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLTl; FOS; FOSLl (FRA-1); FY (DARC); GABRP (GABAa); GAGEBl; GAGECl; GALNAC4S-6ST; GATA3; GDF5; GFIl; GGTl; GMCSF; GNAS1; GNRH1; GPR2 (CCR10); GPR31; GPR44; GPR81 (FKSG80); GRCC10 (CIO); GRP; GSN (Gelsolin); GSTP1; glycoprotein (GP) Ilb/IIIa; HAVCR2; HDAC4; HDAC5; HDAC7A; HDAC9; Her2; HGF; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); L-selectin; LAMA5; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; MIB 1; midkine; MIF; MIP-2; MKI67 (Ki-67); MMP2; MMP9; MS4A1; MSMB; MT3 (metallothionectin-III); MTSSl; MUCl (mucin); MYC; MYD88; NCK2; neurocan; NKG2D; NFKB1; NFKB2; NGF; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NRII2; NRII3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PARTI; PATE; PAWR; PCA3; PCNA; PDGFA; PDGFB; PECAM1; PF4 (CXCL4); PGE2; PGF; PGR; phosphacan; PIAS2; PIK3CG; plasminogen activator; PLAU (uPA); PLG; PLXDC1; PPBP (CXCL7); PPID; PR1; PRKCQ; PRKD1; PRL; PROC; Protein C; PROK2; PSAP; PSCA; PTAFR; PTEN; PTGS2 (COX-2); PTN; RAC2 (p21Rac2); RAGE; RARB; RGSl; RGS13; RGS3; RNFllO (ZNF144); ROB02; SI00A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRR1B (Sprl); ST6GAL1; STAB1; STAT6; STEAP; STEAP2; substance P; TB4R2; TBX21; TCP10; TDGF1; TEK; TGFA; TGFB1; TGFB111; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-a; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSFIO (TRAIL); TNFSF11 (TRANCE); TNFSF12 (AP03L); TNFSF13 (April); TNFSF13B; TNFSF14 (HVEML); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase lia); TP53; TPM1; TPM2; TRADD; TRAF1; TRAF2; TRAF3; TRAF4; TRAF5; TRAF6; TREM1; TREM2; TRPC6; TSLP; TWEAK; thrombomodulin; thrombin; VEGF; VEGFB; VEGFC; versican; VHL C5; VLA-4; XCL1 (lymphotactin); XCL2 (SCM-1b); XCR1 (GPR5/CCXCR1); YY1; and ZFPM2. In another aspect, the tumor target is selected from a tumor targeting antigen, HER1, HER2, HER3, GD2, carcinoembryonic antigens (CEAs), epidermal growth factor receptor active mutant (EGFR VIII), CD133, Fibroblast Activation Protein Alpha (FAP), Epithelial cell adhesion molecular (Epcam), Glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), or a Claudin family protein. In another aspect, the first antigen binding site or the second antigen binding site binds a T-cell marker. In another aspect, the T-cell marker is selected from CTLA-4, PD-1, Lag3, S15, B7H3, B7H4, TCR-alpha, TCR-beta, or TIM-3. In another aspect, the first antigen binding site or the second antigen binding site binds a T-cell activator. In another aspect, the T-cell activator is selected from CD3, 41BB or OX40. In another aspect, the cleavable linker is a protease cleavable linker. In another aspect, the cleavable linker is cleaved by a tumor associated protease: MMP1, MMP2, MMP3, MMP7, MMP9, MMP10, MMP11, MMP12, MMP13, MMP14, MMP15, MMP16, MMP17, MMP19, MMP20, MMP21, uPA, FAPa, or Cathepsin B. In another aspect, the cleavable linker is cleaved by proteases upregulated during apoptosis or inflammation associated responses. In another aspect, the cleavable linker is cleaved by a caspase. In another aspect, the caspase is Caspase 1, Caspase 2, Caspase 3, Caspase 4, Caspase 5, Caspase 6, Caspase 7, Caspase 8, Caspase 9, Caspase 10, Caspase 11 and Caspase 12. In another aspect, the cleavable linker does not mask an antigen binding site. In another aspect, the aAb further comprises an agent conjugated to the aAb. In another aspect, the aAb further comprises a cytokine attached to, or in a fusion protein with the aAb or an Fc region. In another aspect, the cytokine is selected from at least one of: growth hormone; parathyroid hormone; thyroxine; insulin; proinsulin; relaxin; prorelaxin; glycoprotein hormones; hepatic growth factor; fibroblast growth factor; prolactin; placental lactogen; TNF-a; mullerian-inhibiting substance; gonadotropin-associated peptide; inhibin; activin; vascular endothelial growth factor; integrin; thrombopoietin (TPO); nerve growth factors; platelet-growth factor; placental growth factor, transforming growth factors (TGFs); insulin-like growth factor-1 and -11; erythropoietin (EPO); osteoinductive factors; interferons; colony stimulating factors (CSFs); lymphotoxin-alpha; lymphotoxin-beta; CD27L; CD30L; FASL; 4-1 BBL; OX40L; TRAIL; IL-1; IL-2; IL-3; IL-4; IL-5; IL-6; IL-7; IL-8; IL-9; IL-10; IL-11; IL-12; IL-13; IL-15; IL-18; IL-21; IL-22; IL-23; IL-33; IFN-a; IFN-b; IFN-g; IFN-g inducing factor (IGIF); bone morphogenetic protein (BMP); leukemia inhibitory factor (LIF); or kit ligand (KL). In another aspect, the aAb has is selected from SEQ ID NOS: 1, 2, or 3. In another aspect, the agent is at least one of: a toxin or toxic fragment thereof; a microtubule inhibitor; a nucleic acid damaging agent; a detectable moiety; or a diagnostic agent.

In another embodiment, the present invention includes a pharmaceutical composition comprising the activatable Ab. In another embodiment, the present invention includes a method of reducing binding activity of an activatable Ab against normal tissues and targeting a cancer cell comprising administering an effective amount of the activatable Ab to a subject in need thereof. In another embodiment, the present invention includes a method of treating, alleviating a symptom of, or delaying the progression of a cancer comprising administering an effective amount of the activatable Ab a subject in need thereof. In one aspect, the cancer is a cancer that expresses an enzyme that cleaves the cleavable linker. In another aspect, the cancer is selected from a bladder cancer, a bone cancer, a breast cancer, a carcinoid, a cervical cancer, a colon cancer, an endometrial cancer, a glioma, a head and neck cancer, a liver cancer, a lung cancer, a lymphoma, a melanoma, an ovarian cancer, a pancreatic cancer, a prostate cancer, a renal cancer, a sarcoma, a skin cancer, a stomach cancer, a testis cancer, a thyroid cancer, a urogenital cancer, or a urothelial cancer. In another aspect, the cancer is selected from the group consisting of: acute myeloid leukemia, adrenocortical carcinoma, B-cell lymphoma, bladder urothelial carcinoma, breast ductal carcinoma, breast lobular carcinoma, carcinomas of the esophagus, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin's lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung aquamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma.

In another embodiment, the present invention includes an activatable antibody (aAb) comprising, in order, the following structure: a first light chain comprising a first variable light region; a cleavable linker; a first heavy chain comprising a first variable heavy region; and wherein the cleavable linker prevents or reduces the first light chain and the first heavy chain from forming an antigen binding site against a first antigen; wherein cleavage of the cleavable linker releases the first heavy chain to form an antibody binding site with the first light chain that binds a first antigen. In another aspect, the aAb further comprises at least one of a first constant light region or a first constant heavy region, respectively. In another aspect, the aAb further comprises an Fc region attached to a first constant heavy region, wherein the Fc region is a wild-type or a mutant domain that modifies Fc receptor binding, a second variable heavy region and a second Fc region, or a second variable heavy region and a second Fc region and an uncleavable flexible linker and a second variable light region and a second heavy variable region, or a second Fc region and an uncleavable flexible linker and a cytokine. In another aspect, the aAb further comprises a second antibody binding site formed by a second variable light chain and a second constant light chain connected to the first heavy chain that binds a second antigen, and optionally a flexible non-cleavable linker between the second variable light chain and the second constant light chain. In another aspect, the aAb further comprises at least one of a first constant heavy region, a first constant heavy region, or both. In another aspect, the Fc region is a wild-type Fc region, a mutated Fc region, a monomeric wild type Fc region, a monomeric mutant Fc region, a dimeric wild type Fc region, or a dimeric mutant Fc region. In another aspect, the aAb further comprises a cytokine attached to, or in a fusion protein with the aAb or the Fc region. In another aspect, the cytokine is selected from at least one of: growth hormone; parathyroid hormone; thyroxine; insulin; proinsulin; relaxin; prorelaxin; glycoprotein hormones; hepatic growth factor; fibroblast growth factor; prolactin; placental lactogen; TNF-alpha; mullerian-inhibiting substance; gonadotropin-associated peptide; inhibin; activin; vascular endothelial growth factor; integrin; thrombopoietin (TPO); nerve growth factors; platelet-growth factor; placental growth factor, transforming growth factors (TGFs); insulin-like growth factor-1 and -11; erythropoietin (EPO); osteoinductive factors; interferons; colony stimulating factors (CSFs); lymphotoxin-alpha; lymphotoxin-beta; CD27L; CD30L; FASL; 4-1 BBL; OX40L; TRAIL; IL-1; IL-2; IL-3; IL-4; IL-5; IL-6; IL-7; IL-8; IL-9; IL-10; IL-11; IL-12; IL-13; IL-15; IL-18; IL-21; IL-22; IL-23; IL-33; IFN-a; IFN-beta; IFN-gamma; IFN-gamma inducing factor (IGIF); bone morphogenetic protein (BMP); leukemia inhibitory factor (LIF); or kit ligand (KL). In another aspect, the first antigen is a tissue specific surface antigen selected from ICAM1; VCAM1; EpCAM; extra domain B of fibronectin; melanoma-associated chondroitin sulfate proteoglycan (MCSP); melanoma-associated proteoglycan (MAPG); high molecular weight melanoma associated antigen (HMV-MAA); prostate specific membrane antigen (PSMA); epidermal growth factor receptor (EGFR); hepatocyte growth factor receptor (HGFR); fibroblast activation protein (FAP); carcinoembryonic antigen (CEA); cell-adhesion molecule (CAM); human B-cell maturation target (BCMA); placental growth factor (PLGF); folate receptor, insulin-like growth factor receptor (ILGFR); CD133; CD40; CD37; CD33; CD30; CD28; CD24; CD23; CD22; CD21; CD20; CD19; CD13; CD10; HER3; HER2; nonmuscle myosin heavy chain type A (nmMHCA); transferrin; epithelial cell adhesion molecule (EpCAM); annexin A 1; nucleotin, tenascin, vascular endothelial growth factor receptor 1 (VEGFR1), vascular endothelial growth factor receptor 2; (VEGFR-2); aminopeptidase N, tie-1, tie-2, or c-Met. In another aspect, the first antigen is selected from a protein, a portion of a protein, or a peptides encoded by at least one gene selected from: ABCF1; ACVR1; ACVR1B; ACVR2; ACVR2B; ACVRL1; ADORA2A; aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-la); CCL4 (MIP-lb); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-lRB/RA); CCR3 (CKR3/CMKBR3); CCR4; CCR5 (CMKBR5/ChemR13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wapl/Cipl); CDKNIB (p27Kipl); CDKNIC; CDKN2A (p16INK4a); CDKN2B; CDKN2C; CDKN3; CEBPB; CER1; CHGA; CHGB; chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CXCLIO(IP-IO); CXCL11 (I-TAC/IP-9); CXCL12 (SDF1); CXCL13; CXCL14; CXCL16; CXCL2 (GR02); CXCL3 (GR03); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; CGRP; Clq; CIR protein; CI; C4a; C4b; C2a; C2b; C3a; C3b; DAB2IP; DES; DKFZp451J0118; DNCL1; DPP4; E-selectin; E2F1; ECGF1; EDG1; EFNA1; EFNA3; EFNB2; EGF; EGFR; ELAC2; ENG; ENOl; EN02; EN03; EPHB4; EPO; ERBB2 (Her-2); EREG; ERK8; ESR1; ESR2; F3 (TF); Factor VII; Factor IX; Factor V; Factor VIIa; Factor X; Factor XII; Factor XIII; FADD; FasL; FASN; FCER1A; FCER2; Fc gamma receptor; FCGR3A; FGF; FGFl (aFGF); FGF10; FGFll; FGF12; FGF12B; FGFl 3; FGFl 4; FGF16; Fgfl7; Fgfl 8; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRTl (fibronectin); FLTl; FOS; FOSLl (FRA-1); FY (DARC); GABRP (GABAa); GAGEBl; GAGECl; GALNAC4S-6ST; GATA3; GDF5; GFIl; GGTl; GMCSF; GNAS1; GNRH1; GPR2 (CCR10); GPR31; GPR44; GPR81 (FKSG80); GRCC10 (CIO); GRP; GSN (Gelsolin); GSTP1; glycoprotein IIb; glycoprotein IIIa; HAVCR2; HDAC4; HDAC5; HDAC7A; HDAC9; Her2; HGF; HIF1A; HIP1; histamine and histamine receptors; HLA-A; HLA-DRA; HM74; HMGB1; HMOX1; HUMCYT2A; ICEBERG; ICOSL; ID2; IFN-alpha; IFNA1; IFNA2; IFNA4; IFNA5; IFNA6; IFNA7; IFNB1; IFN-gamma; IFNW1; IGBP1; IGF1; IGF1R; IGF2; IGFBP2; IGFBP3; IGFBP6; IL-1; ILIA; IL1B; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; ILIA; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2; IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAKI; IRAK2; ITGA1; ITGA2; ITGA3; ITGA6 (a6 integrin); ITGAV; ITGB3; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); L-selectin; LAMA5; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; MIB1; midkine; MIF; MIP-2; MKI67 (Ki-67); MMP2; MMP9; MS4A1; MSMB; MT3 (metallothionectin-III); MTSSl; MUCl (mucin); MYC; MYD88; NCK2; neurocan; NKG2D; NFKB1; NFKB2; NGF; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NRII2; NRII3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PARTI; PATE; PAWR; PCA3; PCNA; PDGFA; PDGFB; PECAM1; PF4 (CXCL4); PGE2; PGF; PGR; phosphacan; PIAS2; PIK3CG; plasminogen activator; PLAU (uPA); PLG; PLXDC1; PPBP (CXCL7); PPID; PR1; PRKCQ; PRKDl; PRL; PROC; Protein C; PROK2; PSAP; PSCA; PTAFR; PTEN; PTGS2 (COX-2); PTN; RAC2 (p21Rac2); RAGE; RARB; RGSl; RGS13; RGS3; RNFllO (ZNF144); ROB02; SI00A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRR1B (Sprl); ST6GAL1; STAB1; STAT6; STEAP; STEAP2; substance P; TB4R2; TBX21; TCP10; TDGF1; TEK; TGFA; TGFB1; TGFB111; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF-alpha; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSFIO (TRAIL); TNFSF11 (TRANCE); TNFSF12 (AP03L); TNFSF13 (April); TNFSF13B; TNFSF14 (HVEML); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase Iia); TP53; TPM1; TPM2; TRADD; TRAF1; TRAF2; TRAF3; TRAF4; TRAF5; TRAF6; TREM1; TREM2; TRPC6; TSLP; TWEAK; thrombomodulin; thrombin; VEGF; VEGFB; VEGFC; versican; VHL C5; VLA-4; XCL1 (lymphotactin); XCL2 (SCM-1b); XCR1 (GPR5/CCXCR1); YY1; and ZFPM2. In another aspect, the first and second antigen are at least one of: the same antigen; the first and second antigen are different; or the first and second antigen is the same antigen but the first antigen binding site and the second antigen binding site bind different epitopes of the same antigen. In another aspect, the first antigen binding site or the second antigen binding site binds a tumor target. In another aspect, the tumor target is selected from a tumor targeting antigen, HER1, HER2, HER3, GD2, carcinoembryonic antigens (CEAs), epidermal growth factor receptor active mutant (EGFRVIII), CD133, fibroblast Activation Protein Alpha (FAP), epithelial cell adhesion molecular (Epcam), glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), or a Claudin family member. In another aspect, the first antigen binding site or the second antigen binding site binds a T-cell marker. In another aspect, the T-cell marker is selected from CTLA-4, PD-1, Lag3, S15, B7H3, B7H4, TCR-alpha, TCR-beta, TIM-3. In another aspect, the first antigen binding site or the second antigen binding site binds a T-cell activator. In another aspect, the T-cell activator is selected from CD3, 41BB or OX40. In another aspect, the cleavable linker is a protease cleavable linker. In another aspect, the cleavable linker is cleaved by a tumor associated protease: MMP1, MMP2, MMP3, MMP7, MMP9, MMP10, MMP11, MMP12, MMP13, MMP14, MMP15, MMP16, MMP17, MMP19, MMP20, MMP21, uPA, FAPa, or Cathepsin B. In another aspect, the cleavable linker is cleaved by proteases upregulated during apoptosis or inflammation associated responses. In another aspect, the cleavable linker is cleaved by a caspase. In another aspect, the caspase is Caspase 1, Caspase 2, Caspase 3, Caspase 4, Caspase 5, Caspase 6, Caspase 7, Caspase 8, Caspase 9, Caspase 10, Caspase 11 and Caspase 12. In another aspect, the cleavable linker does not mask an antigen binding site. In another aspect, the aAb further comprises an agent conjugated to the aAb. In another aspect, the agent is at least one of: a toxin or toxic fragment thereof; a microtubule inhibitor; a nucleic acid damaging agent; a detectable moiety; or a diagnostic agent. In another aspect, the aAb has is selected from SEQ ID NOS: 1, 2, or 3.

In another embodiment, the present invention includes a nucleic acid that encodes an activatable antibody (aAb) that includes, in order, the following structure: a first light chain comprising: a first variable light region; a cleavable linker; a first heavy chain comprising: a first variable heavy region; wherein the cleavable linker prevents or reduces the first light chain and the first heavy chain from forming a first antigen binding site against a first antigen; and wherein cleavage of the cleavable linker releases the first heavy chain to allow formation of the first antigen binding site to bind a first antigen.

In another embodiment, the present invention includes a nucleic acid that encodes an activatable antibody (aAb) that includes, in order, the following structure: a first light chain comprising a first variable light region; a cleavable linker; a first heavy chain comprising a first variable heavy region; and wherein the cleavable linker prevents or reduces the first light chain and the first heavy chain from forming an antigen binding site against a first antigen;

wherein cleavage of the cleavable linker releases the first heavy chain to form an antibody binding site with the first light chain that binds a first antigen.

In another embodiment, the present invention includes a cell that comprises a nucleic acid that encodes an activatable antibody (aAb) that includes, in order, the following structure: a first light chain comprising: a first variable light region; a cleavable linker; a first heavy chain comprising: a first variable heavy region; wherein the cleavable linker prevents or reduces the first light chain and the first heavy chain from forming a first antigen binding site against a first antigen; and wherein cleavage of the cleavable linker releases the first heavy chain to allow formation of the first antigen binding site to bind a first antigen.

In another embodiment, the present invention includes a cell that comprises a nucleic acid that encodes an activatable antibody (aAb) that includes, in order, the following structure: a first light chain comprising a first variable light region; a cleavable linker; a first heavy chain comprising a first variable heavy region; and wherein the cleavable linker prevents or reduces the first light chain and the first heavy chain from forming an antigen binding site against a first antigen; wherein cleavage of the cleavable linker releases the first heavy chain to form an antibody binding site with the first light chain that binds a first antigen.

In another embodiment, the present invention includes a pharmaceutical composition comprising the activatable Ab and a carrier. In another embodiment, the present invention includes a method of reducing binding activity of an antibody against normal tissues and targeting a cancer cell comprising administering an effective amount of the activatable Ab to a subject in need thereof.

In another embodiment, the present invention includes a method of treating, alleviating a symptom of, or delaying the progression of a cancer comprising administering an effective amount of the activatable Ab to a subject in need thereof. In one aspect, the cancer is a cancer that expresses an enzyme that cleaves the cleavable linker. In another aspect, the cancer is selected from a bladder cancer, a bone cancer, a breast cancer, a carcinoid, a cervical cancer, a colon cancer, an endometrial cancer, a glioma, a head and neck cancer, a liver cancer, a lung cancer, a lymphoma, a melanoma, an ovarian cancer, a pancreatic cancer, a prostate cancer, a renal cancer, a sarcoma, a skin cancer, a stomach cancer, a testis cancer, a thyroid cancer, a urogenital cancer, or a urothelial cancer. In another aspect, the cancer is selected from the group consisting of acute myeloid leukemia, adrenocortical carcinoma, B-cell lymphoma, bladder urothelial carcinoma, breast ductal carcinoma, breast lobular carcinoma, carcinomas of the esophagus, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin's lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung aquamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma.

In another embodiment, the present invention includes an activatable Antibody (aAb), in order, comprising: a first variable light region; a cleavable linker; and a first variable heavy region; and an Fc region; wherein the cleavable linker prevents the first variable light region and the first variable heavy region from forming a first antigen binding site against a first antigen, wherein the cleavable linker does not mask the antigen binding site; and wherein cleavage of the cleavable linker releases the first variable heavy region to allow formation of the first antigen binding site that binds a first antigen.

In another embodiment, the present invention includes a cell that expresses an activatable antibody (aAb) comprising, in order, the following structure: a first light chain comprising: a first variable light region; a cleavable linker; a first heavy chain comprising: a first variable heavy region; wherein the cleavable linker prevents or reduces the first light chain and the first heavy chain from forming a first antigen binding site against a first antigen; and wherein cleavage of the cleavable linker releases the first heavy chain to allow formation of the first antigen binding site to bind a first antigen. In one aspect, the cell is a T cell or a mesenchymal stem cell. In another aspect, the aAb further comprises a transmembrane sequence that anchors the aAb to the surface of a T cell to form a chimeric antigen receptor, wherein the cell is a CAR T cell.

### BRIEF DESCRIPTION OF THE DRAWINGS

For a more complete understanding of the features and advantages of the present invention, reference is now made to the detailed description of the invention along with the accompanying figures and in which:

FIGS. 1A-1C show schematic diagrams of proteins and diagrams to show how Pro antibody designs regains the antigen binding ability after cutting. FIG. 1A. The schematics of AABs. FIG. 1B shows the first type of antigen binding activation. CL and VH is linked with a proteolytic linker sensitive to MMP14. VH is not able to pair with VL to form a stable antigen binding site before cutting. VH is released after cutting with MMP14 and is able to pair with VL to form the antigen binding site. FIG. 1C shows the second type of antigen binding activation. The first Fc and VH is linked with a proteolytic linker sensitive to MMP14. VH is not able to pair with VL to form a stable antigen binding site before cutting. VH is released after cutting with MMP14 and is able to pair with VL to form the antigen binding site.

FIG. 2: Pro anti-CLDN18.2-Fc restores the antigen binding ability after the linker is cut with MMP14. Cell-based ELISA data shows that pro anti-CLDN18.2-Fc after cutting with MMP14 binds to the CLDN18.2 expressing KatoIII cells as strong as the positive control. The binding of uncut protein is about 100 times weaker to the positive control.

FIG. 3: Bi-specific pro anti-CLDN18.2-Fc-anti-hCD3 restores the antigen binding ability after the linker is cut with MMP14. Cell-based ELISA data shows that pro anti-CLDN18.2-Fc-anti-hCD3 after cutting with MMP14 binds to the CLDN18.2 expressing KatoIII cells more than 20 times stronger than that of uncut proteins.

FIG. 4: Bi-specific pro anti-CLDN18.2-Fc-anti-hCD3 after MMP14 cutting activates T cells more than 10 times stronger than that of uncut protein. Reporter Jurkat cells were mixed with KatoIII cells and pro anti-CLDN18.2-Fc-anti-hCD3 with or without MMP14 cutting. After 24 hours incubation, the level of T cell activation was measured via the luciferase production.

FIG. 5: Pro anti-hCTLA4 ScFv-Fc after cutting with MMP14 binds to surface coated hCTLA4 proteins more than 10 times stronger than uncut protein.

FIG. 6: Pro anti-CLDN18.2 clone 2 restores the antigen binding ability after the linker is cut with MMP14. Cell-based ELISA data shows that pro anti-CLDN18.2 clone 2 after cutting with MMP14 binds to the CLDN18.2 expressing KatoIII about 100× stronger than that of uncut protein.

FIG. 7: Pro anti-hCD3 ScFab restores the hCD3e binding ability after the linker is cut with MMP14. ELISA data shows that pro anti-hCD3 ScFab after cutting with MMP14 binds to surface coated hCD3e more than 20× stronger than that of uncut protein.

FIG. 8: Pro anti-hCD3(AAB7) restores the hCD3e binding ability after the linker is cut with MMP14. ELISA data shows that pro anti-hCD3(AAB7) after cutting with MMP14 binds to coated hCD3e more than 10× stronger than that of uncut protein.

FIG. 9: Pro anti-hCTLA4 (AAB7) restores the antigen binding ability after the linker is cut with MMP14. Flow Cytometry-Based Binding Assay data shows that pro anti-hCTLA4 (AAB7) after cutting with MMP14 binds to the hCTLA4 expressing cells as strong as the positive control while the uncut protein shows almost no binding.

FIG. 10: Pro anti-hCD3(AAB7) restores the ability to stimulate T cell activation after the linker is cut with MMP14. Reporter T cell activation assay data shows that pro anti-hCD3(AAB7) after cutting with MMP14 stimulates T cell activation more than 20× stronger than that of uncut protein. The level of T cell activation is measured via the luciferase production.

FIG. 11: Bi-specific Pro anti-hCD3(AAB8)-anti-hPD-L1 restores the ability to stimulate T cell activation after the linker is cut with MMP14. Reporter T cell activation assay data shows that Bi-specific Pro anti-hCD3(AAB8)-anti-hPD-L1 after cutting with MMP14 stimulates T cell activation more than 20× stronger than that of uncut protein. The level of T cell activation is measured via the luciferase production.

FIG. 12: Bi-specific Pro anti-hCD3(AAB8)-anti-CLDN18.2 ScFv restores the ability to stimulate T cell activation after the linker is cut with MMP14. Reporter T cell activation assay data shows that Bi-specific Pro anti-hCD3(AAB8)-anti-CLDN18.2 ScFv after cutting with MMP14 stimulates T cell activation more than 20× stronger than that of uncut protein. The level of T cell activation is measured via the luciferase production.

FIG. 13: Bi-specific Pro anti-hCD3(AAB8)-pro anti-hPD-L1 restores the ability to stimulate T cell activation in PBMC after the linker is cut with MMP14. Bi-specific Pro anti-hCD3(AAB8)-pro anti-hPD-L1 after cutting with MMP14 stimulates T cell activation in PBMC more than 20× stronger than that of uncut protein. The level of T cell activation is measured via the IFN-gamma production.

FIG. 14: Pro anti-hCTLA4 (AAB1)-Fc restores the antigen binding ability after the linker is cut with MMP14. Flow Cytometry-Based Binding Assay data shows that pro anti-hCTLA4 (AAB1)-Fc after cutting with MMP14 binds to the hCTLA4 expressing cells as strong as the positive control while the uncut protein shows almost no binding.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

Therapeutic monoclonal antibodies have been developed to treat a variety of human diseases including cancer. In cancer therapy, for example, anti-CTLA4 antibody or anti-CD3 antibodies have been used to activate T cells via reducing the immunosuppression signal in tumor microenvironment. However, systemic T cell over activation due to off-target Ag-Ab interactions leads to significant adverse events. Antibodies against tumor-associated antigens (TAA) often targets non-tumor tissues that express the same antigens. In the case of anti-CTLA4 activatable antibodies of the prior art, the constructs add both synthetic/foreign extraneous peptides (the masking moiety) in addition to cleavable linkers to block the activity of anti-CTLA-4. These two extra peptides can lead to an immune response to the linker, and further, cleavage of the linker does not ensure release of the masking moiety from the antigen binding site.

The present invention reduces off-target toxicities for monoclonal antibody therapies, thereby increasing the therapeutic index and drug tolerability for patients, by eliminating the use of a masking moiety. It was found that the pro-antibodies taught herein have little to no activity until the drug reaches the tumor, thus yielding a long half-life, protein stability and manufacturability. The new pro-antibodies are designed to shorten heavy and light chains of antibody with a short linker that reduces binding to the targeting molecules. Since this short linker is sensitive to tumor associated proteases, when the linker is cut it restores the topologic position of heavy and light chains and therefore its binding affinity to the target at tumor tissues.

As used herein, the term “activatable antibodies”, “aAb”, “pro-antibody”, or “probody” refers to a fusion protein that includes antibody antigen binding domains that are separated by a cleavable linker. The basic structure of the fusion protein includes, from amino to carboxy: a variable light region-cleavable linker-variable heavy region or a variable heavy region-cleavable linker-variable light region. The first fusion protein can be co-expressed with a second fusion protein that targets a second antigen, while the first fusion protein binds a first antigen. The first and second antigens can be the same antigen, a different antigen, or even the same antigen but bind a different epitope of the antigen. The fusion protein may also include one or more of the following: the constant light region, the constant heavy region, Fc region (wild-type or mutant), a second linker between the Fc and a second protein (e.g., a cytokine).

A nucleic acid encoding the aAb can be part of a vector that is used to express the aAb in a host cell, such as a bacterial, fungal, plant, or mammalian cell.

As used herein, the terms “antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain variable fragment (scFv) antibodies. An antibody other than a “bispecific” or “bifunctional” antibody is understood to have identical binding sites. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

As used herein, the term “bispecific” or “bifunctional” antibody is understood to have two different antigen binding sites. For example, the bispecific antibody of the present invention will include two different antigen binding domains, e.g., a first and a second antigen binding domain that each binds a first and a second antigen, respectively. The bispecific antibody can also have two different antigen binding regions that bind the same antigen, but at two different epitopes. More commonly, the bispecific antibody will bind two different antigens. The first or second antigen will generally be a tumor specific antigen, while the other antigen binding region with bind a T cell activating molecule on a T cell.

As used herein, the term “antibody” is used in the broadest sense, and specifically covers monoclonal antibodies (including full length antibodies or other bivalent, Fc-region containing antibodies such as bivalent scFv Fc-fusion antibodies), polyclonal antibodies, multispecific antibodies (e.g., bispecific antibodies), and antibody fragments (e.g., Fab, Fab′, F(ab′)₂, Fv, scFv) so long as they exhibit the desired biological activity. Antibodies (Abs) and immunoglobulins (Igs) are glycoproteins having the same structural characteristics.

While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas. The present invention includes monoclonal antibodies (and binding fragments thereof) that are completely recombinant, in other words, where the complementarity determining regions (CDRs) are genetically spliced into a human antibody backbone, often referred to as veneering an antibody. Thus, in certain aspects the monoclonal antibody is a fully synthesized antibody. In certain embodiments, the monoclonal antibodies (and binding fragments thereof) can be made in bacterial or eukaryotic cells, including plant cells.

As used herein, the term “antibody fragment” refers to a portion of a full-length antibody, generally the antigen binding or variable region and include Fab, Fab′, F(ab′)₂, Fv and scFv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of cross-linking antigen, and a residual other fragment (which is termed pFc′). As used herein, “functional fragment” with respect to antibodies, refers to Fv, F(ab) and F(ab′)₂ fragments.

As used herein, the “Fv” fragment is the minimum antibody fragment that contains a complete antigen recognition and binding site. This region consists of a dimer of one heavy and one light chain variable domain in a tight, non-covalent association (VH-VL dimer). It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond. While the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985), relevant portions incorporated herein by reference.

As used herein, an “isolated” antibody is one that has been identified and separated and/or recovered from a component of the environment in which it was produced. Contaminant components of its production environment are materials, which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the term “antibody mutant” refers to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95, 96, 97, 98, or 99%.

As used herein, the term “variable” in the context of variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342:877), or both, that is Chothia plus Kabat. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat et al.) The constant domains are not involved directly in binding an antibody to an antigen, but exhibit various effector function, such as participation of the antibody in antibody-dependent cellular toxicity.

The light chains of antibodies (immunoglobulin) from any vertebrate species can be assigned to one of two clearly distinct types, called kappa and lambda, based on the amino sequences of their constant domain.

Depending on the amino acid sequences of the constant domain of their heavy chains, “immunoglobulins” can be assigned to different classes. There are at least five (5) major classes of immunoglobulins: IgA, IgD, IgE, IgG and IgM, and several of these may be further divided into subclasses (isotypes), e.g., IgG-1, IgG-2, IgG-3 and IgG4; IgA-1 and IgA-2. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed and claimed invention may be made by the hybridoma method first described by Kohler and Milstein, Nature 256, 495 (1975), relevant portions incorporated herein by reference.

All monoclonal antibodies utilized in accordance with the presently disclosed and claimed invention will be either (1) the result of a deliberate immunization protocol, as described in more detail herein below; or (2) the result of an immune response that results in the production of antibodies naturally in the course of a disease or cancer.

The uses of the monoclonal antibodies of the presently disclosed and claimed invention may require administration of such or similar monoclonal antibody to a subject, such as a human. However, when the monoclonal antibodies are produced in a non-human animal, such as a rodent or chicken, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed and claimed invention can be “humanized”, that is, the antibodies are engineered such that antigenic portions thereof are removed and like portions of a human antibody are substituted therefore, while the antibodies' affinity for a specific antigen is retained. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, relevant portions incorporated herein by reference.

Humanized forms of antibodies are chimeric immunoglobulins, immunoglobulin chains or fragments thereof (such as Fab, Fab′, F(ab′)₂, Fv, scFv or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting nonhuman (i.e. rodent, chicken) CDRs or CDR sequences for the corresponding sequences of a human antibody. (See also U.S. Pat. No. 5,225,539.) In some instances, Fv framework residues of the human immunoglobulin are replaced by corresponding non-human residues from the donor antibody. Humanized antibodies can also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin.

The aAb of the present invention can also include an engineered sequence or glycosylation sites that confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

The aAb can be a single chain variable fragment (scFv) that is a fusion of the variable regions of the heavy and light chains of immunoglobulins. This chimeric molecule retains the specificity of the original immunoglobulin, despite removal of the constant regions and the introduction of a linker peptide between the two antigen binding domains. This modification usually leaves the specificity unaltered after cleavage of the linker. These molecules were created historically to facilitate phage display where it is highly convenient to express the antigen-binding domain as a single peptide. The scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

The present invention includes activatable antibodies (also referred to as pro-antibodies, or probodies) that target specific antigens. Examples of antigens include first antigen is a tissue specific surface antigen selected from ICAM1; VCAM1; EpCAM; extra domain B of fibronectin; melanoma-associated chondroitin sulfate proteoglycan (MCSP); melanoma-associated proteoglycan (MAPG); high molecular weight melanoma associated antigen (HMV-MAA); prostate specific membrane antigen (PSMA); epidermal growth factor receptor (EGFR); hepatocyte growth factor receptor (HGFR); fibroblast activation protein (FAP); carcinoembryonic antigen (CEA); cell-adhesion molecule (CAM); human B-cell maturation target (BCMA); placental growth factor (PLGF); folate receptor, insulin-like growth factor receptor (ILGFR); CD133; CD40; CD37; CD33; CD30; CD28; CD24; CD23; CD22; CD21; CD20; CD19; CD13; CD10; HER3; HER2; nonmuscle myosin heavy chain type A (nmMHCA); transferrin; epithelial cell adhesion molecule (EpCAM); annexin A 1; nucleotin, tenascin, vascular endothelial growth factor receptor 1 (VEGFR1), vascular endothelial growth factor receptor 2; (VEGFR-2); aminopeptidase N, tie-1, tie-2, or c-Met. Other antigens include a protein, a portion of a protein, or a peptides encoded by at least one gene selected from: ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1; B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-la); CCL4 (MIP-lb); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-lRB/RA); CCR3 (CKR3/CMKBR3); CCR4; CCR5 (CMKBR5/ChemR 13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wapl/Cipl); CDKNIB (p27Kipl); CDKNIC; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CXCLIO(IP-IO); CXCL11 (I-TAC/IP-9); CXCL12 (SDF1); CXCL13; CXCL14; CXCL16; CXCL2 (GR02); CXCL3 (GR03); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; CGRP; Clq; Clr; CI; C4a; C4b; C2a; C2b; C3a; C3b; DAB2IP; DES; DKFZp451J0118; DNCL1; DPP4; E-selectin; E2F1; ECGF1; EDG1; EFNA1; EFNA3; EFNB2; EGF; EGFR; ELAC2; ENG; ENOl; EN02; EN03; EPHB4; EPO; ERBB2 (Her-2); EREG; ERK8; ESR1; ESR2; F3 (TF); Factor VII; Factor IX; Factor V; Factor Vila; Factor Factor X; Factor XII; Factor XIII; FADD; FasL; FASN; FCER1A; FCER2; Fc gamma receptor; FCGR3A; FGF; FGFl (aFGF); FGF10; FGFll; FGF12; FGF12B; FGFl 3; FGFl 4; FGF16; FGFl 7; FGFl 8; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRTl (fibronectin); FLTl; FOS; FOSLI (FRA-1); FY (DARC); GABRP (GABAa); GAGEBl; GAGECl; GALNAC4S-6ST; GATA3; GDF5; GFIl; GGTl; GMCSF; GNAS1; GNRH1; GPR2 (CCR10); GPR31; GPR44; GPR81 (FKSG80); GRCC10 (CIO); GRP; GSN (Gelsolin); GSTP1; glycoprotein (GP) Ilb/IIIa; HAVCR2; HDAC4; HDAC5; HDAC7A; HDAC9; Her2; HGF; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); L-selectin; LAMA5; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; MIB 1; midkine; MIF; MIP-2; MKI67 (Ki-67); MMP2; MMP9; MS4A1; MSMB; MT3 (metallothionectin-III); MTSSl; MUCl (mucin); MYC; MYD88; NCK2; neurocan; NKG2D; NFKB1; NFKB2; NGF; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NRII2; NRII3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PARTI; PATE; PAWR; PCA3; PCNA; PDGFA; PDGFB; PECAM1; PF4 (CXCL4); PGE2; PGF; PGR; phosphacan; PIAS2; PIK3CG; plasminogen activator; PLAU (uPA); PLG; PLXDC1; PPBP (CXCL7); PPID; PR1; PRKCQ; PRKDl; PRL; PROC; Protein C; PROK2; PSAP; PSCA; PTAFR; PTEN; PTGS2 (COX-2); PTN; RAC2 (p21Rac2); RAGE; RARB; RGSl; RGS13; RGS3; RNFllO (ZNF144); ROB02; SI00A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRR1B (Sprl); ST6GAL1; STAB1; STAT6; STEAP; STEAP2; substance P; TB4R2; TBX21; TCP10; TDGF1; TEK; TGFA; TGFB1; TGFB111; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-a; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSFIO (TRAIL); TNFSF11 (TRANCE); TNFSF12 (AP03L); TNFSF13 (April); TNFSF13B; TNFSF14 (HVEML); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase lia); TP53; TPM1; TPM2; TRADD; TRAF1; TRAF2; TRAF3; TRAF4; TRAF5; TRAF6; TREM1; TREM2; TRPC6; TSLP; TWEAK; thrombomodulin; thrombin; VEGF; VEGFB; VEGFC; versican; VHL C5; VLA-4; XCL1 (lymphotactin); XCL2 (SCM-1b); XCR1 (GPR5/CCXCR1); YY1; and/or ZFPM2.

The present invention also includes tumor targeting antigens selected from HER1, HER2, HER3, GD2, carcinoembryonic antigens (CEAs), epidermal growth factor receptor active mutant (EGFRVIII), CD133, Fibroblast Activation Protein Alpha (FAP), Epithelial cell adhesion molecular (Epcam), Glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), or a Claudin family protein.

The present invention also includes antigen binding domains that target T-cell markers. Examples of T-cell marker include CTLA-4, PD-1, Lag3, S15, B7H3, B7H4, TCR-alpha, TCR-beta, and/or TIM-3. The antibodies may also bind to activating T cell markers, CD3, 41BB or OX40.

The present invention also includes cleavable linkers, such as protease cleavable linkers. Examples of cleavable linker are peptides that include sequences cleaved by a tumor associated protease: MMP1, MMP2, MMP3, MMP7, MMP9, MMP10, MMP11, MMP12, MMP13, MMP14, MMP15, MMP16, MMP17, MMP19, MMP20, MMP21, uPA, FAPa, or Cathepsin B. Other examples include a cleavable linker that is cleaved by proteases upregulated during apoptosis or inflammation associated responses, e.g., a caspase. Examples of caspases are Caspase 1, Caspase 2, Caspase 3, Caspase 4, Caspase 5, Caspase 6, Caspase 7, Caspase 8, Caspase 9, Caspase 10, Caspase 11, and/or Caspase 12. Unlike the activatable antibodies of the prior art, the cleavable linker of the present invention does not directly mask an antigen binding site.

The present invention can also include a cytokine with the aAb, e.g., as part of the aAb fusion protein or attached separately to the aAb. The cytokine can be selected from at least one of: growth hormone; parathyroid hormone; thyroxine; insulin; proinsulin; relaxin; prorelaxin; glycoprotein hormones; hepatic growth factor; fibroblast growth factor; prolactin; placental lactogen; TNF-a; mullerian-inhibiting substance; gonadotropin-associated peptide; inhibin; activin; vascular endothelial growth factor; integrin; thrombopoietin (TPO); nerve growth factors; platelet-growth factor; placental growth factor, transforming growth factors (TGFs); insulin-like growth factor-1 and -11; erythropoietin (EPO); osteoinductive factors; interferons; colony stimulating factors (CSFs); lymphotoxin-alpha; lymphotoxin-beta; CD27L; CD30L; FASL; 4-1 BBL; OX40L; TRAIL; IL-1; IL-2; IL-3; IL-4; IL-5; IL-6; IL-7; IL-8; IL-9; IL-10; IL-11; IL-12; IL-13; IL-15; IL-18; IL-21; IL-22; IL-23; IL-33; IFN-a; IFN-b; IFN-g; IFN-g inducing factor (IGIF); bone morphogenetic protein (BMP); leukemia inhibitory factor (LIF); or kit ligand (KL).

The pro-antibody design of the present invention. To avoid the off-target Ag-Ab interaction, the strategy of the present invention is to reduce or even block the antigen binding of antibody via introducing a proteolytic linker to twist the protein structure of the antigen binding site. After the linker is cut at the target site, the two portions of the antibody that, together, form the antigen binding region of the antibody are released from the twisted structure and the antibody regains its antigen binding ability.

The design and method of the fusion protein disclosed herein can be applied to all kinds of antibody without adding extra-elements into the antibody structure. Further, it was found that the short linkers that reduce immunogenicity and high production of antibody. Further, the present invention includes no repeated G4S linkers, thereby reducing the problem with aggregation of the fusion protein prior to cleavage linker.

### Example 1

Materials and Methods. Cloning and protein production.

DNA fragments for protein expression were either synthesized by Genewiz or produced by PCR and cloned into pEE6.4 vector via isothermal assembly (Quantabio).

For protein expression, plasmids were mixed with PEI (Sigma) in 293 Free style medium (Gibco) and transfected into 293F cells. The cells were incubated at 37 °C with shaking at 120 rpm. After a 5~6 day incubation, the supernatant was harvested and filtered. The proteins were purified using Protein A resin (Repligen) and stored in neutralized elution buffer (40 mM Tris pH7.0/100 mM Glycine/100 mM NaCl).

ELISA and Cell-based ELISA. For testing the binding strength of anti-CTLA4 antibodies, CTLA4 proteins (Sino Biological) were diluted to 2 μg/ml and coated on the 96 well plate. Testing antibodies were diluted to different concentrations and added to the wells. After a 1-hour incubation at 37 °C, the unbound protein was washed away with wash buffer (PBS/0.05% Tween 20). The detecting antibody (AP-Goat-anti-human IgG from Jackson ImmunoResearch) was added. After a 1-hour incubation at 37 °C, the unbound protein was washed away with wash buffer. Then the PNPP substrate (Pierce) solution was added. Incubate at room temperature until yellow color develops. OD405 was measured using Biotek Epoch 2 and analyzed using Gen5 software. For testing the binding strength of anti-CLDN18.2 proteins, the method is as above except CLDN18.2 expressing KatoIII cells (ATCC) were coated on the 96 well plate instead of protein. Each well contained 10⁵ cells.

T-cell activation assay. For testing the ability to activate T cell for Pro-anti-CLDN18.2-Fc-CD3 before and after MMP14 cutting, Jurkat NFAT cells (InvivoGen), KatoIII cells (ATCC) and diluted proteins before and after MMP14 cutting were mixed in each well of 96-well plate. Each well contains 104 Jurkat cells and 104 KatoIII cells. After a 30-hour incubation at 37 °C, 30 μl of supernatant was transferred from each well to a black 96 well plate. QUANTI-Luc assay solution (InvivoGen) was then injected to measure luciferase activity using a luminometer (Biotek Synergy) and analyzed using Gen5 software.

Protein sequences
Pro-anti-CTLA4-Fc
(SEQ ID NO: 1)
MINEFSSLAGAQRQRLLGVVVVQSLVHHGAAKHVALLPSALVHCQIASES
KAAVGIQHGHGGLVVVLGLAVAFPFHGDIARVEALHQTAQRHLILGQLVS
AGRLGVHLRLPRLAFGLADGLLNGGGQSLVGDLALVLLAVEPVLVQHGQH
GHHSICGVVLLLSGLGFGVVHFDAVHVPVKLHLGVLMADVHHHACHLGCS
RDHQCILGFGREQKHGRSAQQFGSRTARNRYHRALTPIVEVAGLTRTVIS
RGVLGGYRVNLQKELVFRGVTGDRDTRFDRRVVIGRTFIIDETHPFNFLT
WELTDPIPTITGGDGGAGNRARQAERTGRINQTRTRFFQFYLSAYVLTPP
FDFQLGARTERVRIVVPLLAVVKRLVFVLNRRNGQREVGTRARTTETVRN
AGVTGRRVVDQQFRRLTRLLHVPIQIVGFRVRVEQALRAFARDGRFFTRR
HAQRRWRLGHHNVSGGTWNPEQQYP
Pro-anti-CLDN18.2-Fc
(SEQ ID NO: 2)
METDTLLLWVLLLWVPGSTGDIVMTQSPDSLAVSLGERATISCKSSQSLL
NSGNQKNYLTWYQQKPGQPPKLLIYWASTRESGVPDRFSGSGSGTDFTLT
ISSLQAEDVAVYYCQNDYFYPFTFGQGTKLEIKRTVAAPSVFIFPPSDEQ
LKSGTASVVCLLNNFYPREAKVQWKVDNALQSGNSQESVTEQDSKDSTYS
LSSTLTLSKADYEKHKVYACEVTHQGLSSPVTKSFNRGECGSSGRSENIR
TAGGSQVQLVQSGAEVKKPGSSVKVSCKASGYAFSNYLIEWVKQAPGQGL
EWIGLINPGSGGTNYNEKFKGKATITADKSTSTAYMELSSLRSEDTAVYY
CARVYYGNSFAYWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PGK
Pro-anti-CLDN18.2-Fc-CD3
(SEQ ID NO: 3)
METDTLLLWVLLLWVPGSTGDIVMTQSPDSLAVSLGERATISCKSSQSLL
NSGNQKNYLTWYQQKPGQPPKLLIYWASTRESGVPDRFSGSGSGTDFTLT
ISSLQAEDVAVYYCQNDYFYPFTFGQGTKLEIKRTVAAPSVFIFPPSDEQ
LKSGTASVVCLLNNFYPREAKVQWKVDNALQSGNSQESVTEQDSKDSTYS
LSSTLTLSKADYEKHKVYACEVTHQGLSSPVTKSFNRGECGSSGRSENIR
TAGGSQVQLVQSGAEVKKPGSSVKVSCKASGYAFSNYLIEWVKQAPGQGL
EWIGLINPGSGGTNYNEKFKGKATITADKSTSTAYMELSSLRSEDTAVYY
CARVYYGNSFAYWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPEAAGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALGAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PGKGGGGSGGGGSGGGGSGGGGSQIVLTQSPAIMSASPGEKVTMTCSASS
SVSYMNWYQQKSGTSPKRWIYDTSKLASGVPAHFRGSGSGTSYSLTISGM
EAEDAATYYCQQWSSNPFTFGSGTKLEINGGGGSGGGGSGGGGSQVQLQQ
SGAELARPGASVKMSCKASGYTFTRYTMHWVKQRPGQGLEWIGYINPSRG
YTNYNQKFKDKATLTTDKSSSTAYMQLSSLTSEDSAVYYCARYYDDHYCL
DYWGQGTTLTVSS

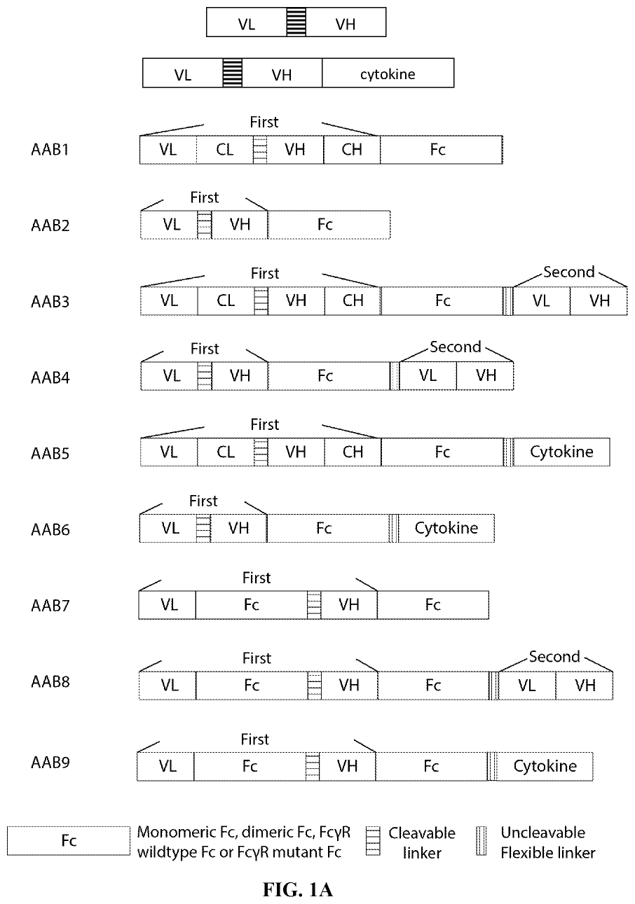

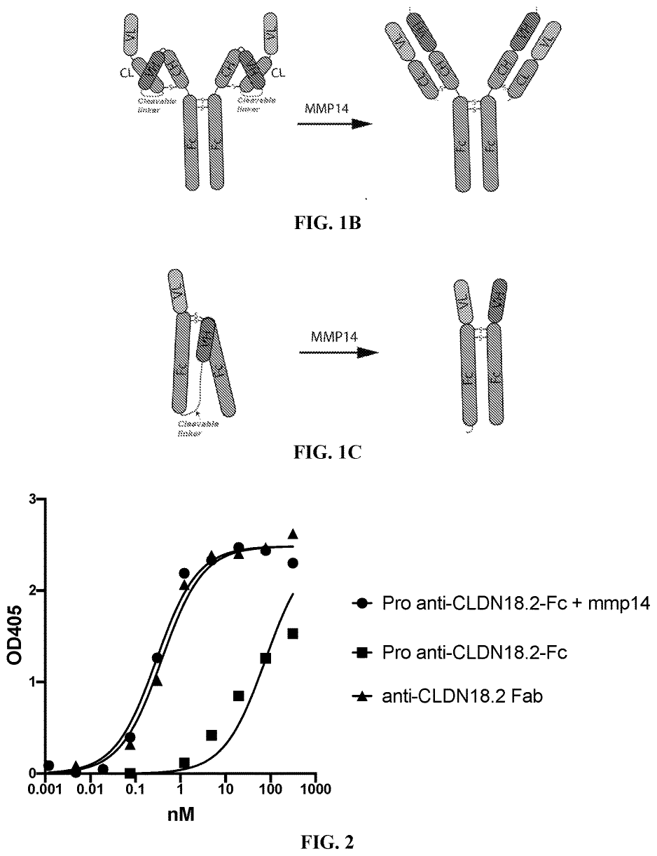

FIGS. 1A-1C show schematic diagrams of proteins and diagrams to show how Pro antibody designs regains the antigen binding ability after cutting. FIG. 1A. The schematics of AABs. FIG. 1B shows the first type of antigen binding activation. CL and VH is linked with a proteolytic linker sensitive to MMP14. VH is not able to pair with VL to form a stable antigen binding site before cutting. VH is released after cutting with MMP14 and is able to pair with VL to form the antigen binding site. FIG. 1C shows the second type of antigen binding activation. The first Fc and VH is linked with a proteolytic linker sensitive to MMP14. VH is not able to pair with VL to form a stable antigen binding site before cutting. VH is released after cutting with MMP14 and is able to pair with VL to form the antigen binding site.

FIG. 2 is a graph that shows a Pro anti-CLDN18.2-Fc restores the antigen binding ability after the linker is cut with MMP14. Cell-based ELISA data shows that pro anti-CLDN18.2-Fc after cutting with MMP14 binds to the CLDN18.2 expressing KatoIII cells as strong as the positive control. The binding of uncut protein is about 100 times weaker to the positive control.

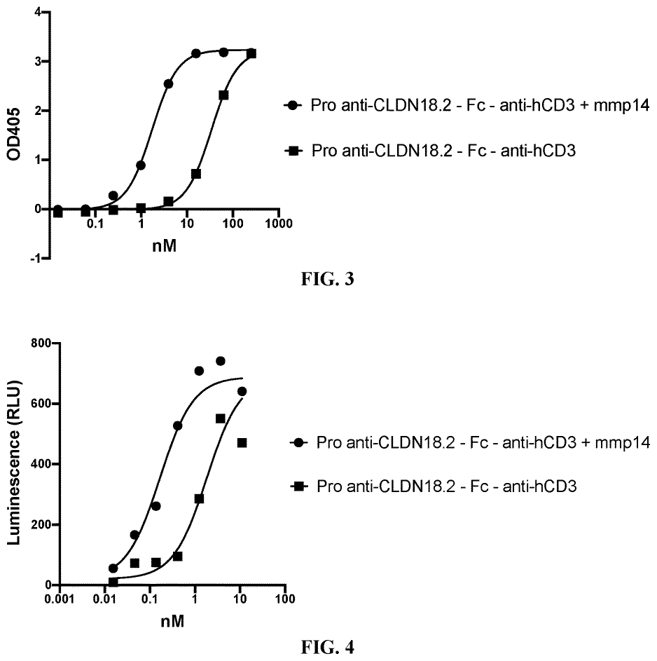

FIG. 3 is a graph that shows a Bi-specific pro anti-CLDN18.2-Fc-anti-hCD3 restores the antigen binding ability after the linker is cut with MMP14. Cell-based ELISA data shows that pro anti-CLDN18.2-Fc-anti-hCD3 after cutting with MMP14 binds to the CLDN18.2 expressing KatoIII cells more than 20 times stronger than that of uncut proteins.

FIG. 4 is a graph that shows a Bi-specific pro anti-CLDN18.2-Fc-anti-hCD3 after MMP14 cutting activates T cells more than 10 times stronger than that of uncut protein. Reporter Jurkat cells were mixed with KatoIII cells and pro anti-CLDN18.2-Fc-anti-hCD3 with or without MMP14 cutting. After 24 hours incubation, the level of T cell activation was measured via the luciferase production.

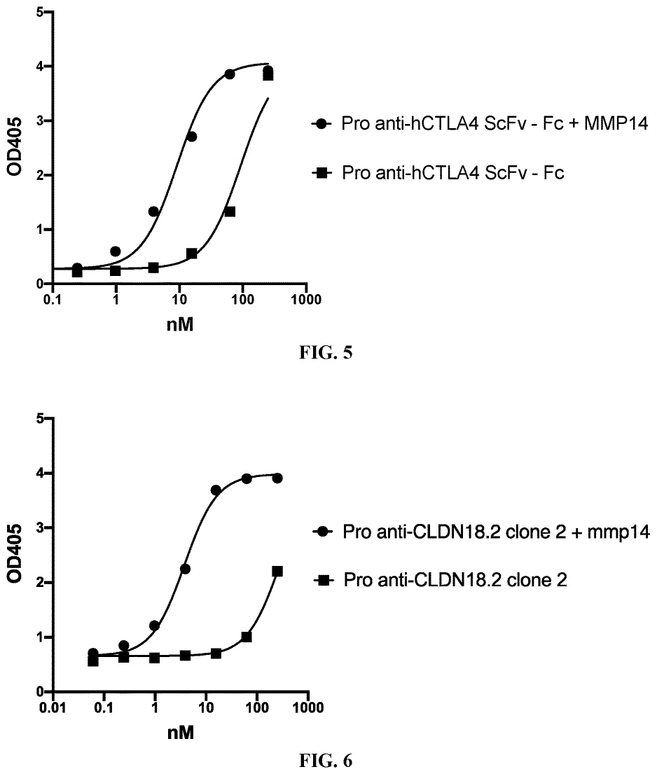

FIG. 5 is a graph that shows a Pro anti-hCTLA4 ScFv-Fc after cutting with MMP14 binds to surface coated hCTLA4 proteins more than 10 times stronger than uncut protein.

FIG. 6 is a graph that shows a Pro anti-CLDN18.2 clone 2 restores the antigen binding ability after the linker is cut with MMP14. Cell-based ELISA data shows that pro anti-CLDN18.2 clone 2 after cutting with MMP14 binds to the CLDN18.2 expressing KatoIII about 100× stronger than that of uncut protein.

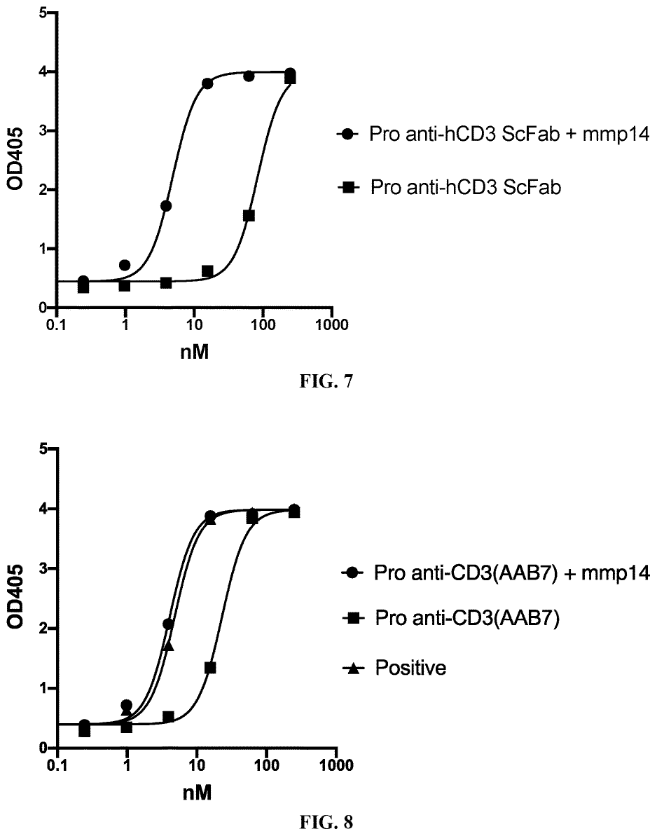

FIG. 7 is a graph that shows a Pro anti-hCD3 ScFab restores the hCD3e binding ability after the linker is cut with MMP14. ELISA data shows that pro anti-hCD3 ScFab after cutting with MMP14 binds to surface coated hCD3e more than 20× stronger than that of uncut protein.

FIG. 8 is a graph that shows a Pro anti-hCD3(AAB7) restores the hCD3e binding ability after the linker is cut with MMP14. ELISA data shows that pro anti-hCD3(AAB7) after cutting with MMP14 binds to coated hCD3e more than 10× stronger than that of uncut protein.

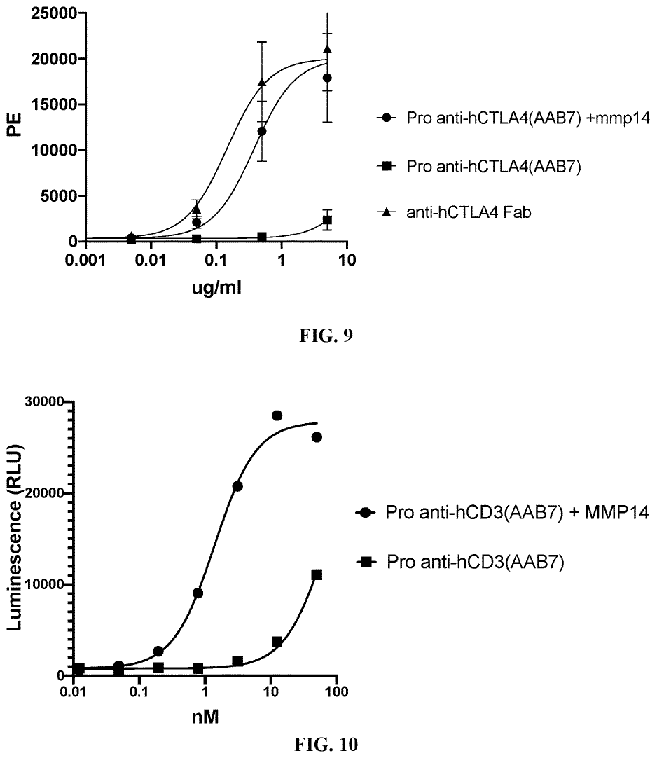

FIG. 9 is a graph that shows a Pro anti-hCTLA4 (AAB7) restores the antigen binding ability after the linker is cut with MMP14. Flow Cytometry-Based Binding Assay data shows that pro anti-hCTLA4 (AAB7) after cutting with MMP14 binds to the hCTLA4 expressing cells as strong as the positive control while the uncut protein shows almost no binding.

FIG. 10 is a graph that shows a Pro anti-hCD3(AAB7) restores the ability to stimulate T cell activation after the linker is cut with MMP14. Reporter T cell activation assay data shows that pro anti-hCD3(AAB7) after cutting with MMP14 stimulates T cell activation more than 20× stronger than that of uncut protein. The level of T cell activation is measured via the luciferase production.

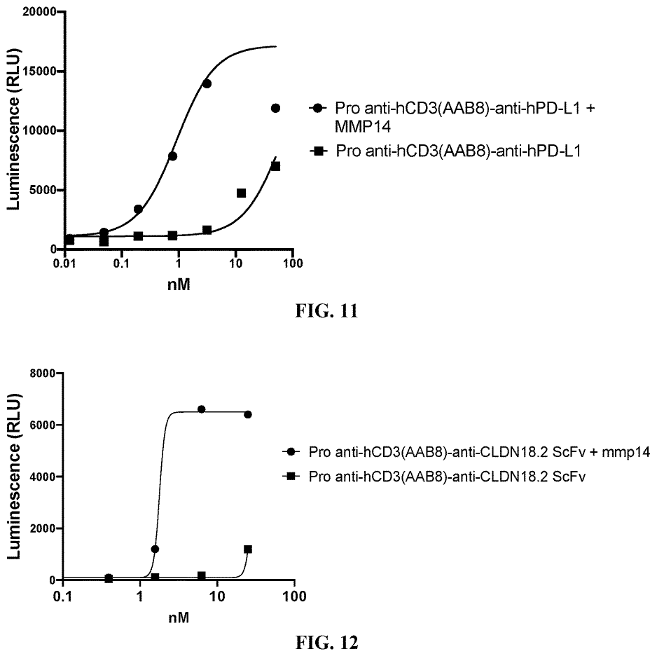

FIG. 11 is a graph that shows a Bi-specific Pro anti-hCD3(AAB8)-anti-hPD-L1 restores the ability to stimulate T cell activation after the linker is cut with MMP14. Reporter T cell activation assay data shows that Bi-specific Pro anti-hCD3(AAB8)-anti-hPD-L1 after cutting with MMP14 stimulates T cell activation more than 20× stronger than that of uncut protein. The level of T cell activation is measured via the luciferase production.

FIG. 12 is a graph that shows a Bi-specific Pro anti-hCD3(AAB8)-anti-CLDN18.2 ScFv restores the ability to stimulate T cell activation after the linker is cut with MMP14. Reporter T cell activation assay data shows that Bi-specific Pro anti-hCD3(AAB8)-anti-CLDN18.2 ScFv after cutting with MMP14 stimulates T cell activation more than 20× stronger than that of uncut protein. The level of T cell activation is measured via the luciferase production.

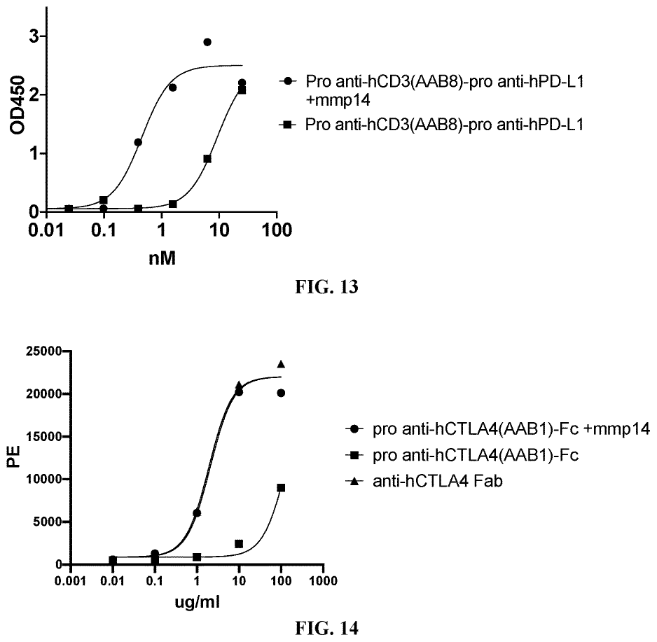

FIG. 13 is a graph that shows a Bi-specific Pro anti-hCD3(AAB8)-pro anti-hPD-L1 restores the ability to stimulate T cell activation in PBMC after the linker is cut with MMP14. Bi-specific Pro anti-hCD3(AAB8)-pro anti-hPD-L1 after cutting with MMP14 stimulates T cell activation in PBMC more than 20× stronger than that of uncut protein. The level of T cell activation is measured via the IFN-gamma production.

FIG. 14 is a graph that shows a Pro anti-hCTLA4 (AAB1)-Fc restores the antigen binding ability after the linker is cut with MMP14. Flow Cytometry-Based Binding Assay data shows that pro anti-hCTLA4 (AAB1)-Fc after cutting with MMP14 binds to the hCTLA4 expressing cells as strong as the positive control while the uncut protein shows almost no binding.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A method of reducing binding activity of an antibody against normal tissues and targeting a cancer cell, treating, alleviating a symptom of, or delaying the progression of a cancer comprising administering an effective amount of an activatable antibody (aAb) to a subject in need thereof;
wherein the aAb comprises, in order, the following structure:
a first light chain comprising:
a first variable light region;
a cleavable linker; and
a first heavy chain comprising:
a first variable heavy region;
wherein the cleavable linker prevents or reduces the first variable heavy region from pairing with the first variable light region to form a first antigen binding site against a first antigen; and
wherein cleavage of the cleavable linker releases the first variable heavy region to pair with the first variable light region to form the first antigen binding site to bind a first antigen.

2. The method of claim 1, wherein the aAb comprises, in order, the following structure:
a first light chain comprising:
a first variable light region and constant light region;
a first heavy chain consisting of:
a first variable heavy region, a constant heavy region, and an Fc region; and
a cleavable linker between the constant light region and the first variable heavy region, wherein the cleavable linker prevents or reduces the first variable heavy region from pairing with the first variable light region to form a first antigen binding site against a first antigen; and
wherein cleavage of the cleavable linker releases the first variable heavy region to pair with the first variable light region to form the first antigen binding site to bind a first antigen.

3. The method of claim 1, wherein the aAb comprises, in order, the following structure:
a first light chain comprising a first variable light region and a constant light region;
a cleavable linker; and
a first heavy chain comprising a first variable heavy region and a constant heavy region;
wherein the cleavable linker prevents or reduces the first variable heavy region from pairing with the first variable light region to form a first antigen binding site against a first antigen; and
wherein cleavage of the cleavable linker releases the first variable heavy region to pair with the first variable light region to form the first antigen binding site to bind a first antigen.

4. The method of claim 1, wherein the aAb comprises, in order, the following structure:
a first variable light region;
a cleavable linker;
a first variable heavy region; and
an Fc region;
wherein the cleavable linker prevents or reduces the first variable heavy region from pairing with the first variable light region to form a first antigen binding site against a first antigen, wherein the cleavable linker does not mask the antigen binding site; and
wherein cleavage of the cleavable linker releases the first variable heavy region to pair with the first variable light region to form the first antigen binding site to bind a first antigen.

5. The method of claim 1, the aAb does not contain a masking moiety that blocks an antigen binding site.

6. The method of claim 1, wherein the cancer is a cancer that expresses an enzyme that cleaves the cleavable linker.

7. The method of claim 1, wherein:
the cancer is selected from a bladder cancer, a bone cancer, a breast cancer, a carcinoid, a cervical cancer, a colon cancer, an endometrial cancer, a glioma, a head and neck cancer, a liver cancer, a lung cancer, a lymphoma, a melanoma, an ovarian cancer, a pancreatic cancer, a prostate cancer, a renal cancer, a sarcoma, a skin cancer, a stomach cancer, a testis cancer, a thyroid cancer, a urogenital cancer, or a urothelial cancer; or
the cancer is selected from the group consisting of: acute myeloid leukemia, adrenocortical carcinoma, B-cell lymphoma, bladder urothelial carcinoma, breast ductal carcinoma, breast lobular carcinoma, carcinomas of the esophagus, castration-resistant prostate cancer (CRPC), cervical carcinoma, cholangiocarcinoma, chronic myelogenous leukemia, colorectal adenocarcinoma, colorectal cancer (CRC), esophageal carcinoma, gastric adenocarcinoma, glioblastoma multiforme, head and neck squamous cell carcinoma, Hodgkin's lymphoma/primary mediastinal B-cell lymphoma, hepatocellular carcinoma (HCC), kidney chromophobe carcinoma, kidney clear cell carcinoma, kidney papillary cell carcinoma, lower grade glioma, lung adenocarcinoma, lung aquamous cell carcinoma, melanoma (MEL), mesothelioma, non-squamous NSCLC, ovarian serous adenocarcinoma, pancreatic ductal adenocarcinoma, paraganglioma & pheochromocytoma, prostate adenocarcinoma, renal cell carcinoma (RCC), sarcoma, skin cutaneous melanoma, squamous cell carcinoma of the head and neck, T-cell lymphoma, thymoma, thyroid papillary carcinoma, uterine carcinosarcoma, uterine corpus endometrioid carcinoma and uveal melanoma.

8. The method of claim 1, wherein the aAb further comprises a second antigen binding site formed by a second variable light chain and a second variable heavy chain connected to the first heavy chain that binds a second antigen, and optionally a flexible non-cleavable linker between the second variable light chain and the second variable heavy chain.

9. The method of claim 1, wherein the aAb further comprises at least one of a first constant light region, a first constant heavy region, or both.

10. The method of claim 1, wherein the first light chain, the first heavy chain, or both, further comprise:
an Fc region, a wild-type Fc region, a mutated Fc region, a monomeric wild type Fc region, a monomeric mutant Fc region, a dimeric wild type Fc region, or a dimeric mutant Fc region,
or a second variable heavy region and a second Fc region, or a second Fc region and an uncleavable flexible linker and a second variable light region and a second variable heavy region, or a second Fc region and an uncleavable flexible linker and a cytokine, or an uncleavable flexible linker and a cytokine, or an Fc region and an uncleavable flexible linker and a second variable light region and a second variable heavy region, or an Fc region and an uncleavable flexible linker and a cytokine.

11. The method of claim 8, wherein the first and second antigen are at least one of:
(i) the same antigen; the first and second antigen are different;
(ii) the first and second antigen is the same antigen but the first antigen binding site and the second antigen binding site bind different epitopes of the same antigen;
(iii) the first antigen binding site or the second antigen binding site binds a tumor target;
(iv) the first antigen is a tissue specific surface antigen selected from ICAM1; VCAM1; EpCAM; extra domain B of fibronectin; melanoma-associated chondroitin sulfate proteoglycan (MCSP); melanoma-associated proteoglycan (MAPG); high molecular weight melanoma associated antigen (HMV-MAA); prostate specific membrane antigen (PSMA); epidermal growth factor receptor (EGFR); hepatocyte growth factor receptor (HGFR); fibroblast activation protein (FAP); carcinoembryonic antigen (CEA); cell-adhesion molecule (CAM); human B-cell maturation target (BCMA); placental growth factor (PLGF); folate receptor, insulin-like growth factor receptor (ILGFR); CD133; CD40; CD37; CD33; CD30; CD28; CD24; CD23; CD22; CD21; CD20; CD19; CD13; CD10; HER3; HER2; nonmuscle myosin heavy chain type A (nmMHCA); transferrin; epithelial cell adhesion molecule (EpCAM); annexin A 1; nucleotin, tenascin, vascular endothelial growth factor receptor 1 (VEGFR1), vascular endothelial growth factor receptor 2; (VEGFR-2); aminopeptidase N, tie-1, tie-2, or c-Met;
(v) the first antigen is selected from a protein, a portion of a protein, or a peptides encoded by at least one gene selected from: ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1; B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-lb); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR 13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wapl/Cipl); CDKNIB (p27Kipl); CDKNIC; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CXCLIO(IP-IO); CXCL11 (I-TAC/IP-9); CXCL12 (SDF1); CXCL13; CXCL14; CXCL16; CXCL2 (GR02); CXCL3 (GR03); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; CGRP; Clq; Clr; CI; C4a; C4b; C2a; C2b; C3a; C3b; DAB2IP; DES; DKFZp451J0118; DNCL1; DPP4; E-selectin; E2F1; ECGF1; EDG1; EFNA1; EFNA3; EFNB2; EGF; EGFR; ELAC2; ENG; ENOl; EN02; EN03; EPHB4; EPO; ERBB2 (Her-2); EREG; ERK8; ESR1; ESR2; F3 (TF); Factor VII; Factor IX; Factor V; Factor Vila; Factor X; Factor XII; Factor XIII; FADD; FasL; FASN; FCER1A; FCER2; Fc gamma receptor; FCGR3A; FGF; FGFl (aFGF); FGF10; FGFll; FGF12; FGF12B; FGFl 3; FGFl 4; FGF16; FGFl 7; FGFl 8; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; FOS; FOSLl (FRA-1); FY (DARC); GABRP (GABAa); GAGEB1; GAGEC1; GALNAC4S-6ST; GATA3; GDF5; GFIl; GGT1; GMCSF; GNAS1; GNRH1; GPR2 (CCR10); GPR31; GPR44; GPR81 (FKSG80); GRCC10 (CIO); GRP; GSN (Gelsolin); GSTP1; glycoprotein (GP) Ilb/IIIa; HAVCR2; HDAC4; HDAC5; HDAC7A; HDAC9; Her2; HGF; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); L-selectin; LAMA5; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; MIB 1; midkine; MIF; MIP-2; MKI67 (Ki-67); MMP2; MMP9; MS4A1; MSMB; MT3 (metallothionectin-III); MTSS1; MUC1 (mucin); MYC; MYD88; NCK2; neurocan; NKG2D; NFKB1; NFKB2; NGF; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NRII2; NRII3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PARTI; PATE; PAWR; PCA3; PCNA; PDGFA; PDGFB; PECAM1; PF4 (CXCL4); PGE2; PGF; PGR; phosphacan; PIAS2; PIK3CG; plasminogen activator; PLAU (uPA); PLG; PLXDC1; PPBP (CXCL7); PPID; PR1; PRKCQ; PRKDl; PRL; PROC; Protein C; PROK2; PSAP; PSCA; PTAFR; PTEN; PTGS2 (COX-2); PTN; RAC2 (p21Rac2); RAGE; RARB; RGSl; RGS13; RGS3; RNFllO (ZNF144); ROB02; SI00A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRR1B (Sprl); ST6GAL1; STAB1; STAT6; STEAP; STEAP2; substance P; TB4R2; TBX21; TCP10; TDGF1; TEK; TGFA; TGFB1; TGFB111; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-a; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSFIO (TRAIL); TNFSF11 (TRANCE); TNFSF12 (AP03L); TNFSF13 (April); TNFSF13B; TNFSF14 (HVEML); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase Iia); TP53; TPM1; TPM2; TRADD; TRAF1; TRAF2; TRAF3; TRAF4; TRAF5; TRAF6; TREM1; TREM2; TRPC6; TSLP; TWEAK; thrombomodulin; thrombin; VEGF; VEGFB; VEGFC; versican; VHL C5; VLA-4; XCL1 (lymphotactin); XCL2 (SCM-1b); XCR1 (GPR5/CCXCR1); YY1; and ZFPM2; (vi) the first antigen binding site or the second antigen binding site binds a T-cell activator; (vii) the first antigen binding site or the second antigen binding site binds a T-cell marker.

12. The method of claim 11, wherein the tumor target is selected from a tumor targeting antigen, HER1, HER2, HER3, GD2, carcinoembryonic antigens (CEAs), epidermal growth factor receptor active mutant (EGFRVIII), CD133, Fibroblast Activation Protein Alpha (FAP), Epithelial cell adhesion molecular (Epcam), Glypican 3 (GPC3), EPH Receptor A4 (EphA), tyrosine-protein kinase Met (cMET), IL-13Ra2, microsomal epoxide hydrolase (mEH), MAGE, Mesothelin, MUC16, MUC1, prostate stem cell antigen (PSCA), Wilms tumor-1 (WT-1), or a Claudin family protein.

13. The method of claim 11, wherein the T-cell activator is selected from CD3, 41BB or OX40.

14. The method of claim 11, wherein the T-cell marker is selected from CTLA-4, PD-1, Lag3, S15, B7H3, B7H4, TCR-alpha, TCR-beta, or TIM-3.

15. The method of claim 1, wherein the cleavable linker is selected from one of the following:
(i) the cleavable linker is a protease cleavable linker;
(ii) the cleavable linker is cleaved by a tumor associated protease: MMP1, MMP2, MMP3, MMP7, MMP9, MMP10, MMP11, MMP12, MMP13, MMP14, MMP15, MMP16, MMP17, MMP19, MMP20, MMP21, uPA, FAPa, or Cathepsin B;
(iii) the cleavable linker is cleaved by proteases upregulated during apoptosis or inflammation associated responses;
(iv) the cleavable linker is cleaved by a caspase;
(v) the cleavable linker is cleaved by a caspase, wherein the caspase is Caspase 1, Caspase 2, Caspase 3, Caspase 4, Caspase 5, Caspase 6, Caspase 7, Caspase 8, Caspase 9, Caspase 10, Caspase 11 or Caspase 12.

16. The method of claim 1, wherein the cleavable linker does not mask an antigen binding site.

17. The method of claim 1, wherein the aAb further comprises:
(i) an agent conjugated to the aAb; or
(ii) a cytokine attached to, or in a fusion protein with the aAb or an Fc region; or
wherein the cytokine is selected from at least one of: growth hormone; parathyroid hormone; thyroxine; insulin; proinsulin; relaxin; prorelaxin; glycoprotein hormones; hepatic growth factor; fibroblast growth factor; prolactin; placental lactogen; TNF-a; mullerian-inhibiting substance; gonadotropin-associated peptide; inhibin; activin; vascular endothelial growth factor; integrin; thrombopoietin (TPO); nerve growth factors; platelet-growth factor; placental growth factor, transforming growth factors (TGFs); insulin-like growth factor-1 and -11; erythropoietin (EPO); osteoinductive factors; interferons; colony stimulating factors (CSFs); lymphotoxin-alpha; lymphotoxin-beta; CD27L; CD30L; FASL; 4-1 BBL; OX40L; TRAIL; IL-1; IL-2; IL-3; IL-4; IL-5; IL-6; IL-7; IL-8; IL-9; IL-10; IL-11; IL-12; IL-13; IL-15; IL-18; IL-21; IL-22; IL-23; IL-33; IFN-a; IFN-b; IFN-g; IFN-g inducing factor (IGIF); bone morphogenetic protein (BMP); leukemia inhibitory factor (LIF); or kit ligand (KL).

18. The method of claim 17, wherein the agent is at least one of: a toxin or toxic fragment thereof; a microtubule inhibitor; a nucleic acid damaging agent; a detectable moiety; or a diagnostic agent.

19. The method of claim 1, wherein the aAb comprises an amino acid sequence selected from SEQ ID NOS: 1, 2, or 3.

20. The method of claim 1, wherein the aAb further comprises a pharmaceutically acceptable diluent or carrier.


SEQUENCE LISTING (3 sequences)

SEQ ID NO: 1 - protein, 475 aa, synthetic construct
  REGION 1..475: note = Synthetic peptide
MINEFSSLAGAQRQRLLGVVVVQSLVHHGAAKHVALLPSALVHCQIASESKAAVGIQHGHGGLVVVLGLAVAFPFHGDIARVEALHQTAQRHLILGQLVSAGRLGVHLRLPRLAFGLADGLLNGGGQSLVGDLALVLLAVEPVLVQHGQHGHHSICGVVLLLSGLGFGVVHFDAVHVPVKLHLGVLMADVHHHACHLGCSRDHQCILGFGREQKHGRSAQQFGSRTARNRYHRALTPIVEVAGLTRTVISRGVLGGYRVNLQKELVFRGVTGDRDTRFDRRVVIGRTFIIDETHPFNFLTWELTDPIPTITGGDGGAGNRARQAERTGRINQTRTRFFQFYLSAYVLTPPFDFQLGARTERVRIVVPLLAVVKRLVFVLNRRNGQREVGTRARTTETVRNAGVTGRRVVDQQFRRLTRLLHVPIQIVGFRVRVEQALRAFARDGRFFTRRHAQRRWRLGHHNVSGGTWNPEQQYP

SEQ ID NO: 2 - protein, 703 aa, synthetic construct
  REGION 1..703: note = Synthetic peptide
METDTLLLWVLLLWVPGSTGDIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSSGRSENIRTAGGSQVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 963 aa, synthetic construct
  REGION 1..963: note = Synthetic peptide
METDTLLLWVLLLWVPGSTGDIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSSGRSENIRTAGGSQVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS